(19) United States
(12) Patent Application Publication
(10) Pub. No.: US 2024/0415677 A1
(43) Pub. Date: Dec. 19, 2024

(54) Prosthetic Liner Textile with Differentiated Knit Fabric Exterior Incorporating Low Extensibility Strips and Patches

(71) Applicants: Alps South Europe, s.ro., Pizen (CZ); Aldo Laghi, Pinellas Park, FL (US)

(72) Inventors: Aldo Laghi, Pinellas Park, FL (US); Nathaniel Vint, Palm Harbor, FL (US)

(21) Appl. No.: 18/752,253

(22) Filed: Jun. 24, 2024

Related U.S. Application Data
(63) Continuation-in-part of application No. 18/734,704, filed on Jun. 5, 2024.
(63) Continuation-in-part of application No. 18/208,289, filed on Jun. 11, 2023.
(63) Continuation-in-part of application No. 18/120,465, filed on Mar. 13, 2023.
(63) Continuation-in-part of application No. 17/181,110, filed on Feb. 22, 2021, now abandoned.
(63) Continuation-in-part of application No. 17/094,885, filed on Nov. 11, 2020, now abandoned.
(60) Provisional application No. 62/942,388, filed on Dec. 2, 2019.
(60) Provisional application No. 62/938,483, filed on Nov. 21, 2019.

(51) Int. Cl. A61F 2/78; A61F 2/50

(52) U.S. Cl. CPC A61F 2/7812; A61F 2002/5009

(57) ABSTRACT

A prosthetic liner having a lower longitudinal stretch in the distal region than in either the proximal or optional intermediate region. The distal region may stretch anywhere from 0-30% vertically and 10-200% horizontally as compared to the proximal region's vertical stretch of 55-125% and 100-175% horizontally. A variety of stitches may be used at the distal end to implement this lowered longitudinal stretch. The liner also has an interior layer of elastomer gel. The design of this prosthetic liner is primarily to prevent the “pistoning” of the amputee's residual limb within the liner and for comfort over pressure-sensitive areas of the residual limb. The liner also includes the use of a low extensibility material which is adhered to the fabric portion of a prosthetic liner or orthotic liner either internally or externally and limits the longitudinal movement while allowing for transversal movement.

### CROSS-REFERENCE TO RELATED APPLICATIONS

This application is a continuation-in-part to pending application Ser. No. 18/734,704 titled “Knit Prosthetic Liner Textile with Differentiated Knit Fabric Exterior Incorporating Low Extensibility Strips,” filed Jun. 5, 2024, which is a continuation-in-part of application Ser. No. 18/208,289 titled “Knit Prosthetic Liner Textile with Differentiated Knit Fabric Exterior Incorporating Low Extensibility Strips,” filed Jun. 11, 2023, which is a continuation-in-part to pending application Ser. No. 18/120,465 titled “Knit Prosthetic Liner Textile with Differentiated Knit Fabric Exterior Incorporating Low-Extensibility Strips,” filed Mar. 13, 2023, which is a continuation-in-part of application Ser. No. 17/181,110 titled “Knit Prosthetic Liner Textile with Differentiated Knit Fabric Exterior Incorporating Low Extensibility Strips,” filed Feb. 22, 2021, which is a continuation-in-part of application Ser. No. 16/120,929 titled “Knit Prosthetic Liner Textile with Differentiated Knit Fabric Exterior,” filed Sep. 4, 2018 (which issued as U.S. Pat. No. 10,966,845 on Apr. 6, 2021) and claims priority to U.S. Provisional Patent Application No. 62/554,102, filed Sep. 5, 2017, the disclosures of which are hereby incorporated by reference herein. This application is also a continuation-in-part of application Ser. No. 17/094,885 titled “Low Extensibility Strips for Prosthetic and Orthotic Application,” filed Nov. 11, 2020, which claims priority to U.S. Provisional Patent Application No. 62/938,483 filed Nov. 21, 2019 and to U.S. Provisional Patent Application No. 62/942,388 filed Dec. 2, 2019, the contents of which are incorporated by reference herein in their entirety.

### BACKGROUND OF THE INVENTION

### Field of the Invention

This invention relates to liners for use in a prosthetic assembly. More particularly, the described invention relates to liners having a particular stitching such that the distal end of the liner has less stretch than the proximal end of the liner so as to reduce pistoning and which incorporates low extensibility strips which reduce elongation in the longitudinal direction when applied to prosthetics and orthotics.

### Description of the Background Art

Prosthetic liners have been in use since the 1970's, mostly custom made, and made of various materials. Silicone liners have been used since the 1980s in the prosthetic industry such as those described in U.S. Pat. No. 4,923,474 granted to Klasson and Kristinsson. Other examples of such liners include U.S. Pat. No. 5,728,168 to Laghi et al., U.S. Pat. No. 5,830,237 granted to Kania, U.S. Pat. No. 5,507,834 to Laghi et al., U.S. Pat. No. 5,443,525 to Laghi et al., and U.S. Pat. No. 5,728,168 to Laghi et al. Gel and urethane liners have also been used for prosthetic and orthotic purposes and, for the most part, have a fabric covering. The fabric covering is used to reinforce the underlying material (silicone, gel, urethane) and allows for extensibility to make it easier to don and doff the liner by rolling it on and off the residual limb

The liners disclosed in U.S. Pat. Nos. 5,443,525, 5,507,234, 5,728,168, 6,544,292, and 6,764,631 each represent advances in the field of fabric covered liners and the disclosures of which are hereby incorporated by reference. Additionally, U.S. Pat. No. 6,454,812, also hereby incorporated by reference, describes a liner comprising additional features attached to the liner textile to limit the vertical stretch at the distal end of a liner and is incorporated herein by reference. This method has been used successfully within the field of prosthetics to provide the liner textile with a strengthened distal end, means for attaching a threaded mechanical feature, and a boundary layer that prevents the thermoplastic elastomer from undesirably passing through the textile during high pressure processing.

Fabric liners having a lower longitudinal elongation at the distal end were initially popular because they prevented “pistoning.” Pistoning is the loss of suspension of a residual limb when weight is taken off of it and the resulting pushing down of the limb on the socket when weight is applied, like the piston of car. This means that every time the amputee attempted to move his/her leg, there would be a delay between when the residual limb moved and when the prosthesis moved. Pistoning is more pronounced in locking liners, i.e. liners that attach the prosthesis to the residual limb by means of a distal umbrella and distal pin with a corresponding lock. Older versions of fabric liners with lower longitudinal elongation at the distal end prevent pistoning but also constrain the knee flexion of the amputee. If a liner exhibits a high longitudinal elongation throughout its length, then the prosthesis will move up and down during ambulation. On the other hand, if there is no longitudinal elongation then the amputee will find it difficult to flex the knee.

In order to overcome this issue, liners with a distal matrix were developed such as the liner described in U.S. Pat. No. 6,454,812, described above. These liners include an additional fabric matrix which is knit to minimal longitudinal elongation where the matrix is bonded to the interior of the external fabric of the liner. However, this construction is expensive to construct due to the fact that the additional steps of applying adhesive to the interior of the exterior fabric, applying the reinforcing matrix to the adhesive layer, and curing of the adhesive are required. The present invention overcomes this problem by eliminating the reinforcing matrix and replaces it with a differentiated knitting of the exterior fabric.

There exists a need for liners with limited extensibility in localized regions of the residual limb and greater extensibility in other regions of the residual limb. Liners such as those described in U.S. Pat. No. 9,216,099 to Laghi, U.S. Pat. Nos. 9,364,347, 8,394,150, 8,852,291, 8,246,694, 8,808,294, 8,226,732, 8,357,206, 6,764,631, 6,544,292, 6,454,812, 5,728,168 and 5,507,834 each describe liners which highlight such a need.

U.S. Pat. No. 6,231,617 to Fay, which is incorporated herein by reference, describes a liner which incorporates elongate arms of a strip or ribbon shape. However, a limitation of Fay is that the arms have to radiate from the distal attachment plate or mounted to a peripheral edge of the distal attachment plate. The present invention allows for the use of low extensibility strips anywhere on or in the prosthetic/orthotic without being limited to a certain location on the device.

Generally, the distal end of locking liners requires the fabric to be less extensible in the longitudinal direction or that a polymer or other solid implement be used in order to reduce pistoning of the prosthetic implement during ambulation or movement. Other regions may benefit from reduced extensibility of the fabric cover such as in the case the above the knee amputees who may exhibits unusually fleshy residual limbs.

The present invention utilizes stitching with reduced stretch regions such that the distal end of the liner has less stretch than the proximal end so as to prevent pistoning.

In addition, prosthetic lock systems that are used to retain a patient in a socket are prone to distracting the distal end of the limb due to a singular attachment point at the distal peak. Utilizing a reinforcement to control the stretch characteristics of the liner help support the limb and prevent skin breakdown due to movement of the tissue or redundant tissue by reducing fluid movement and build up within the limb. Distal distraction is well known within the art to cause skin break down and vascular issues due to the milking effect the liner can have on the residual limb especially if redundant tissue is at the distal end of the limb. Prevention of distal distraction is heavily desired for high activity patients to reduce the tissue from moving away from the residual limb.

Therefore, it is an object of this invention to provide an improvement which overcomes the aforementioned inadequacies of the prior art devices and provides an improvement which is a significant contribution to the advancement of the liner art.

Another object of the invention is to provide a liner for prosthetic assemblies that reduces pistoning of the residual limb when in use.

Another object of the invention is to provide a liner for prosthetic assemblies that can also be used with a locking prosthetic assembly.

Another object of the invention is to provide a liner that implements two or more stitching types.

Another object of the invention is to provide a liner with less stretch capability at the distal end than at the proximal end.

Another object of the invention is to provide a liner with variable stretch characteristics based on knit construction and stitching.

Another object of the invention is to provide strips or other shapes of low extensibility material which can be applied to the exterior of fabric liners.

Another object of the invention is to provide low extensibility material which, when applied to fabric liners, reduce extensibility in the longitudinal direction while maintaining extensibility in the transversal direction.

Another object of the invention is provide a fast and inexpensive way to reduce the extensibility of fabric liners in desired regions.

Another object of the invention is to provide a single-piece strip that controls distal distraction of a residual limb while allowing for anterior and posterior stretch to accommodate the residual limb.

Another object of the invention is to provide a customizable solution for patient comfort such that a prosthetist can adhere appropriate shapes in appropriate locations in order to support or contain a region or segment of the residual limb as required by patient clinical conditions.

Another object of the invention is to contain the dynamic deformation of the residual limb during ambulation.

Another object of the invention is to provide a low extensibility material which can be cut into custom or standard shapes.

Another object of the invention is to prevent distal pistoning is a locking style liner by controlling the distraction of the liner from the tissue.

Another object of the invention is to provide customized patches for amputees with residual/redundant tissue that allow for volume change of the limb.

Another object of the invention is to provide protective panels for sensitive scar tissue areas by creating stable containment pressure.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to a liner for use in a prosthetic assembly having variable stretch regions at a distal end providing different stretch regions incorporating low extensibility strips.

Specifically, the present invention relates to a prosthetic liner having a lower longitudinal stretch in the distal region than in either the proximal or optional intermediate region. The distal region may stretch anywhere from 0-30% vertically and 10-200% horizontally as compared to the proximal region's vertical stretch of 55-125% and 100-175% horizontally. The stretch of the liner at various pressure sensitive regions of a residual limb can also be lowered or heightened depending on the area. A variety of stitches may be used at the distal end to implement this lowered longitudinal stretch. Preferably, the liner is made of a stretchable material. The liner also has an interior layer of elastomer gel. The design of this prosthetic liner is primarily to prevent the “pistoning” of the amputee's residual limb within the liner and for comfort over pressure-sensitive areas of the residual limb. The liner also includes the use of a low extensibility material which is adhered to the fabric portion of a prosthetic liner or orthotic liner either internally or externally and limits the longitudinal movement while allowing for transversal movement. This strip preferably extends from the distal end about 4-12 inches, preferably 5-7 inches, and is 8-24 inches in length, preferably 10-14 inches, and 0.5-4 inches in width in order to control distal distraction. The customizable nature of the invention allows for shaping and personalization depending on the needs of the user. The strip can also aid in donning the liner or adjusting the liner because the vertical stretch is eliminated such that the amputee can pull on the strip area to adjust the liner while in the socket or during initial donning.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a cross-sectional view of the present invention for use with a locking prosthetic assembly comprising two different types of stitches showing the different relevant regions;

FIG. 2 is a cross-sectional view of the present invention showing below-the-knee amputation pressure sensitive areas;

FIG. 3 is a cross-sectional view of the present invention showing below-the-knee-amputation pressure tolerant areas;

FIG. 4 is a cross-sectional view of the present invention showing above-the-knee amputation pressure sensitive areas;

FIG. 5 is a cross sectional view of the present invention showing above-the-knee amputation pressure tolerant areas;

FIGS. 6A-6N are various stitching types that can be used in the distal region of the present invention to prevent pistoning;

FIG. 7 is a cross-sectional view of a liner incorporating the low extensibility material internally;

FIG. 8 is a top view of the low extensibility material in one preferred shape;

FIG. 9 is a top view of the low extensibility material in a second preferred shape;

FIG. 10 is a top view of the low extensibility material in a third preferred shape and configuration;

FIG. 11 is a top view of the low extensibility material in a second preferred configuration;

FIG. 12 is a cross-sectional view of a liner with an alternative arrangement of low extensibility material placed either internally or externally;

FIG. 13 is a cross-sectional view with an alternative arrangement of low extensibility material placed either internally or externally;

FIG. 14 is a perspective view of a wrist orthotic with the low extensibility material adhered externally;

FIG. 15 is a perspective view of a back orthotic with the low extensibility material adhered externally;

FIG. 16A is a perspective view of an ankle orthotic with the low extensibility material adhered externally;

FIG. 16B is a cross-sectional view of the ankle orthotic shown in FIG. 16A;

FIG. 17 is a perspective view of a knee orthotic with the low extensibility material adhered externally;

FIG. 18 is a perspective view of an elbow orthotic with the low extensibility material adhered externally;

FIG. 19 is a cross-sectional view of an alternative embodiment of a liner incorporating the low extensibility material internally;

FIG. 20 is a cross-sectional view of an alternative embodiment of a liner incorporating the low extensibility material internally;

FIG. 21 is a perspective view of the present invention incorporating a sealing element.

Similar reference numerals refer to similar parts throughout the several views of the drawings.

### DETAILED DESCRIPTION OF THE PREFERRED EMBODIMENT

The following description is of the best mode presently contemplated for carrying out the invention. This description is not to be taken in a limiting sense, but is made merely for the purpose of describing one or more preferred embodiments of the invention. The scope of the invention should be determined with reference to the claims.

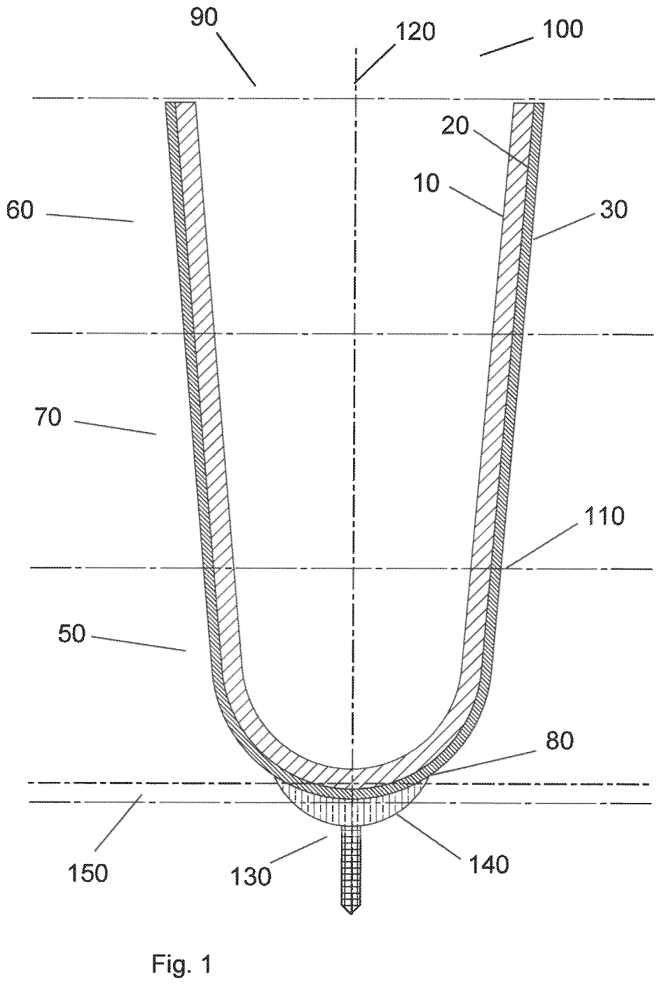

The present invention relates to a liner 100 for use with prosthetic devices. As shown in FIG. 1, the liner 100 for use with a prosthetic assembly comprises a plurality of types of stitching. The liner 100 comprises a distal region 50, proximal region 60, and an optional intermediate region 70. In one embodiment, the distal region 50 that comprises the distal end 80 of the liner is constructed with a reduced vertical stretch when compared to the upper region 60 of the liner. The distal region 50 has a terminal edge 600 that is located at least four inches from the closed distal end. Preferably, the distal region 80 has a vertical stretch of 0-30% from a resting position and a horizontal stretch of 10-200% from a resting position as compared to the proximal region 60, which preferably has a vertical stretch of 55-125% from a resting position and a horizontal stretch of 100-175% from a resting position. As used herein, “stretch” and “elongation” are considered synonymous. Furthermore, “resting position” as used herein means that the liner has not been donned by a patient yet.

Optionally, the intermediate region 70 may have a vertical stretch of 15-40% and a horizontal stretch of 80-120% or it may have the same stretch characteristics of the proximal region 60. The reduced stretch of the distal region 50 is achieved on a flatbed knitting machine by use of a variety of different stitches as described below. In a preferred embodiment, the liner 100 is knit in a single piece construction with the proximal end 90 being open and the distal end 80 being closed. Alternatively, the two halves may be independently constructed and then sewn or attached together by another means to form the completed liner textile.

Preferably, the liner 100 is knit using computerized flatbed knitting machines that allow the use of several different yarns at the same time or sequentially in the same garment, including elastomeric fibers such as Lycra, latex, and silicone among others. These machines also allow the use of different stitch types in different areas of the same garment and controls the tension of each yarn being knitted. Further, an interior gel layer 10 resides on a limb face 22 of exterior fabric layer 30. The gel layer 10 may be any form of stretchable elastomer as known in the industry or later developed but is preferably a styrene-based polymer.

In another embodiment, the liner 100 comprises regions of differing horizontal and vertical stretch stitching depending on anatomical features related to the residual limb or mechanical features related to the prosthetic socket. This means that the boundary 110 between the distal region 50 and the proximal region 60 (or intermediate region 70 if present) may shift location. The boundary 110 is preferably generally perpendicular to the central axis 120 of the liner 100. Additionally, the distal end 70 may house a locking mechanism 130. The use of a locking mechanism 130 requires a construction that is strong enough for the distal attachment plate 140 to be retained by the fabric or else a catastrophic failure of the prosthesis may result. Typically, such locking mechanisms 130 are secured to the fabric through the use of a grommet that bites into the fabric or other similar means of attachment. In order for the fabric to withstand the localized stress imparted by the grommet, the construction of the knit at the distal end must be heavier and stronger than the rest of the fabric cover. Such construction cannot be used in the intermediate region 70 or proximal region 60 because it would impart poor functionality to the liner and limit the user's movement.

More specifically, the fabric in the locking mechanism region 150 must have little to no longitudinal elongation but high circumferential elongation in order to comfortably accommodate the residual limb. The locking mechanism region 150 is limited to the area juxtaposed to the locking mechanism 130. The thickness of the fabric in the locking mechanism region 150 should greater than the thickness of the fabric in the distal region 50. The distal region 50, as compared to the locking mechanism region 150, must have a greater degree of longitudinal elongation in order to facilitate knee flexion. The intermediate region 70 may have an even greater degree of longitudinal elongation and circumferential elongation than the distal region 50 to provide greater comfort to the amputee when sitting or moving.

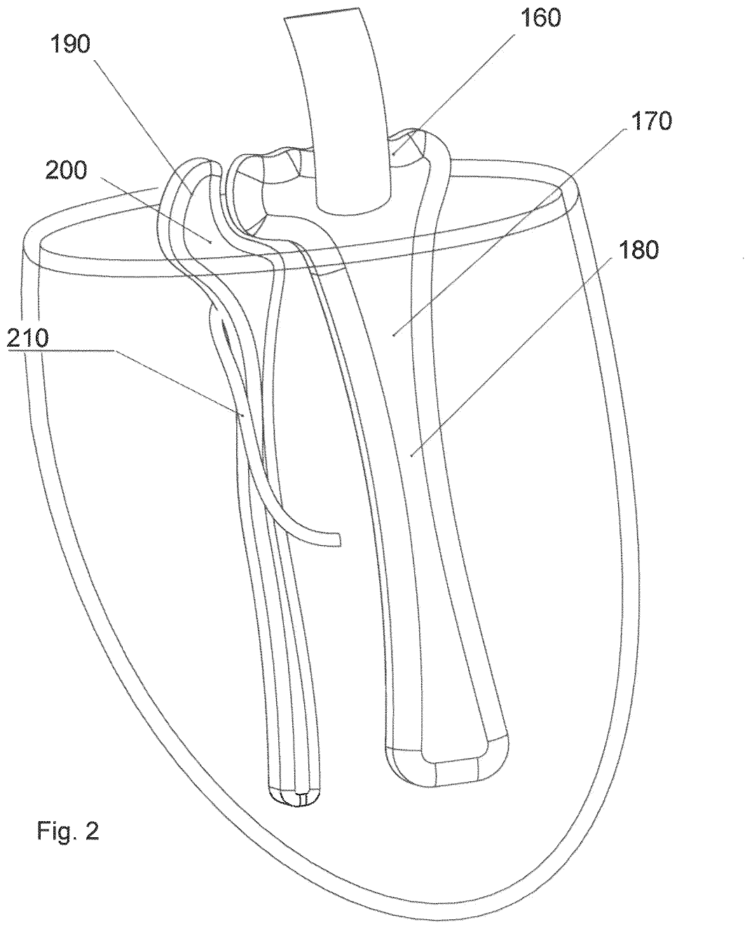

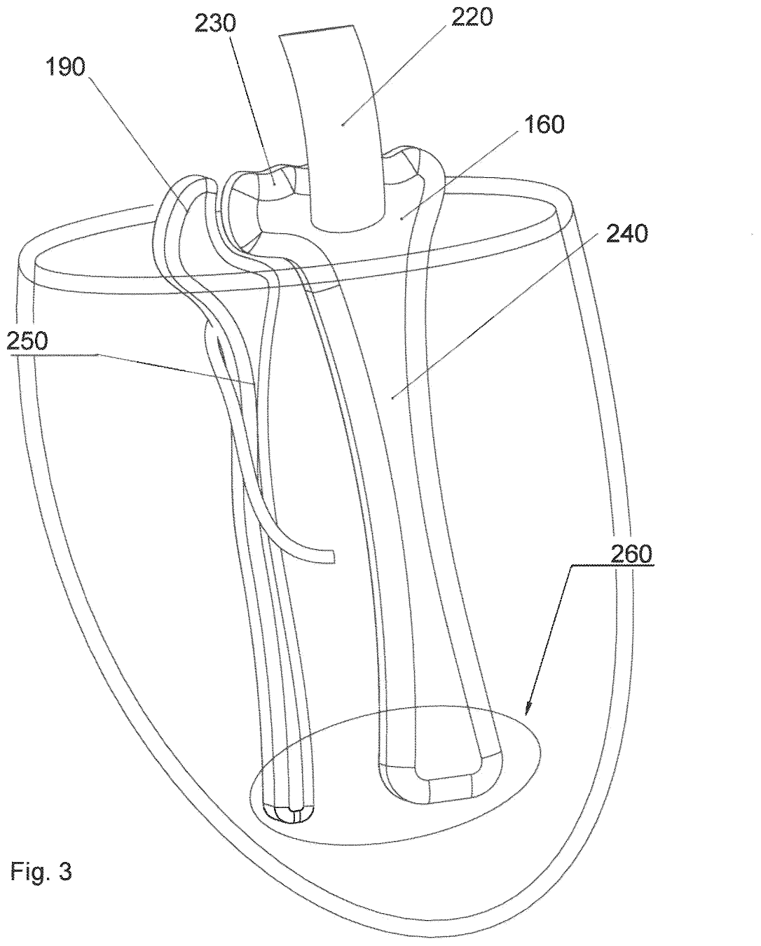

In another embodiment, the liner textile comprises regions of differing horizontal and vertical stretch depending on anatomical features related to the residual limb or mechanical features related to the prosthetic socket. There are various areas of the lower limbs that are more pressure sensitive, requiring more elongation, and various areas that are more pressure tolerant, which require less elongation. As shown in FIG. 2, the pressure sensitive areas for below-the-knee amputations comprise the portions of the tibia 160 such as the anterior tibia 170 and the anterior tibial crest 180 as well as parts of the fibula 190 such as the fibular head and neck 200, and the fibular nerve 210. These regions of the body exhibit sensitivity to pressure and therefore require a liner having greater longitudinal or circumferential elongation at those points. On the other hand, as shown in FIG. 3, the pressure-tolerant areas for below-the-knee amputations comprise the patellar tendon 220, the medial tibia plateau 230, the tibial shaft 240, the fibular shaft 250, and the distal end 260 of tibia 160 and fibula 190. These regions may have more restricted longitudinal or circumferential elongation due to the tolerance these regions exhibit.

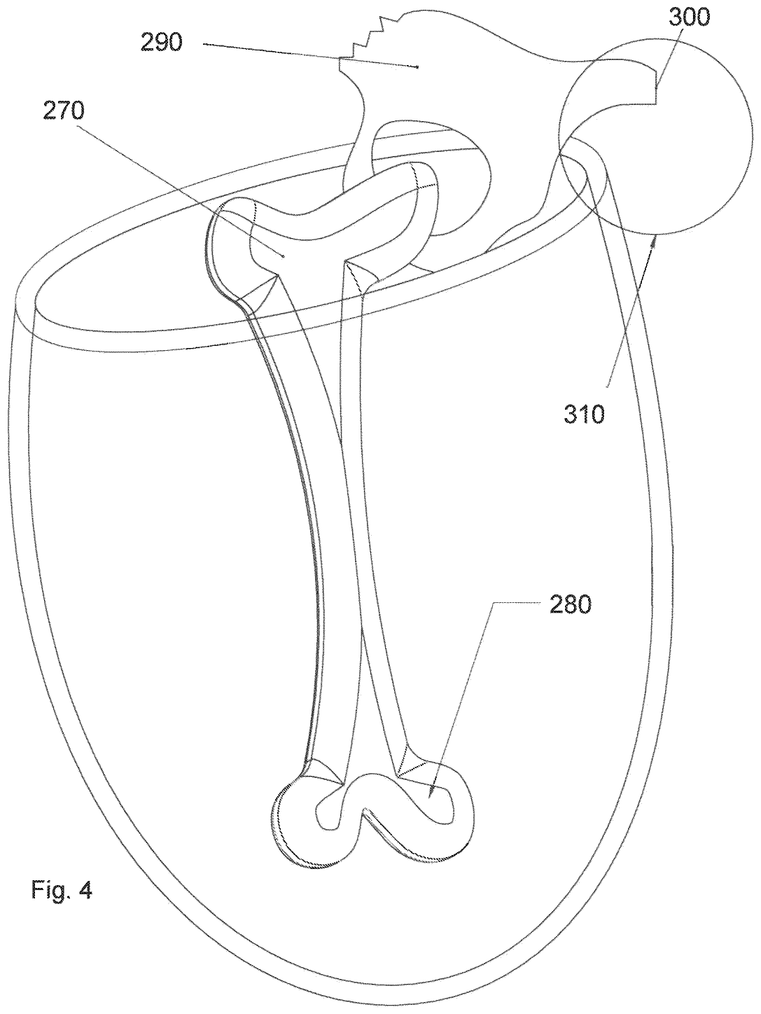

Similarly, as shown in FIGS. 4 and 5, above-the-knee amputations have similarly pressure sensitive and pressure tolerant areas. The pressure sensitive areas comprise the distolateral end 280 of the femur 270, the pubic symphysis 300 of the pelvic bone 290, and the perineal area 310. The pressure tolerant areas comprise the ischial tuberosity 320 of the pelvic bone 290, the gluteals 330, the lateral sides 340 of the residual limb, and the distal end 350 of the femur 270. As with below-the-knee, pressure sensitive areas of above-the-knee amputations require greater longitudinal or circumferential elongation at those points while the pressure tolerant areas may have more restricted longitudinal or circumferential elongation due to the tolerance those regions exhibit.

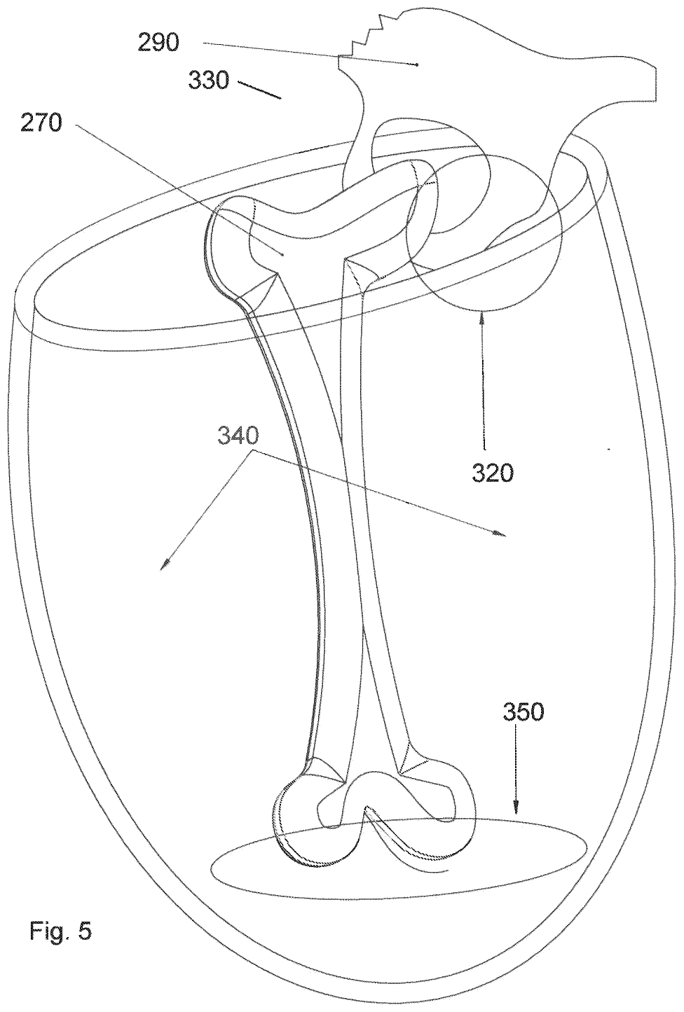

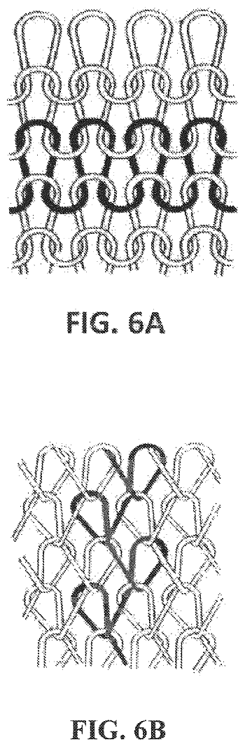

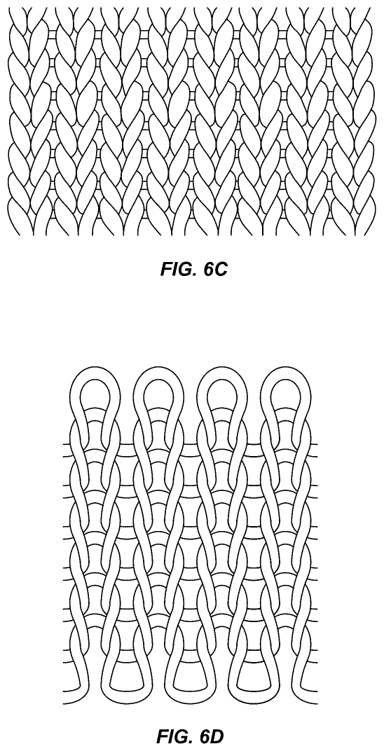

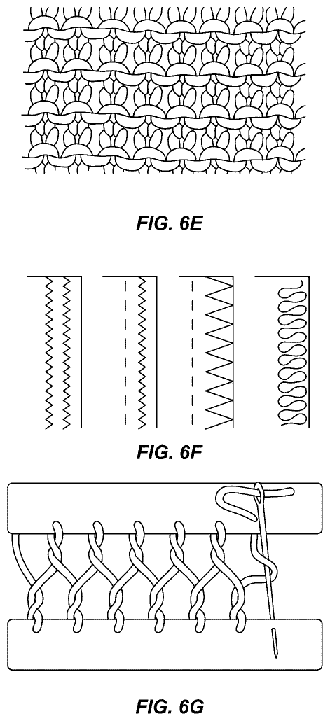

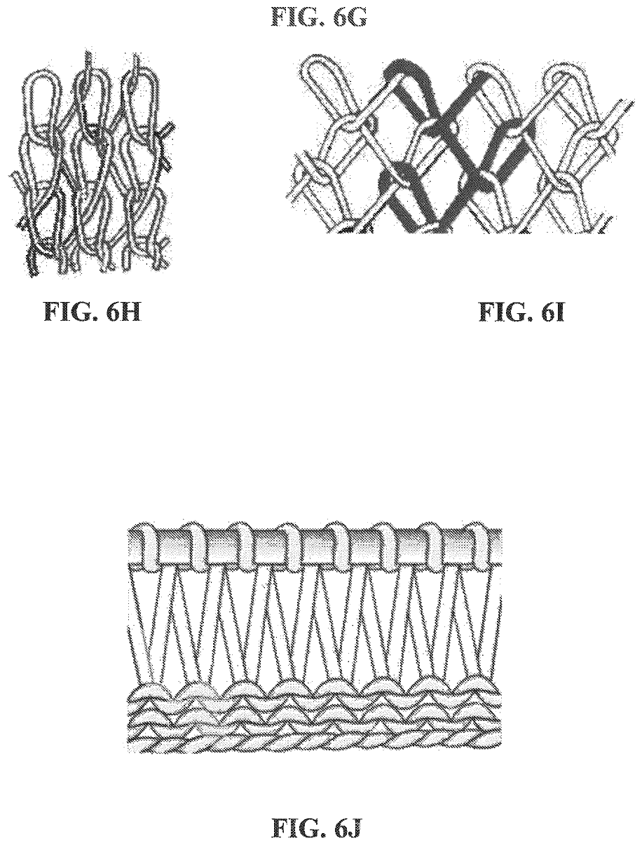

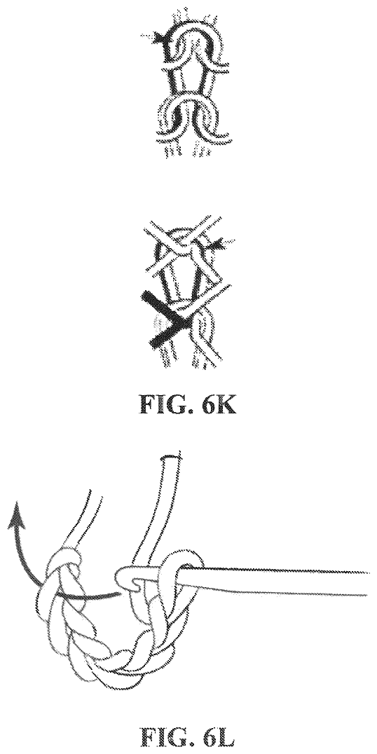

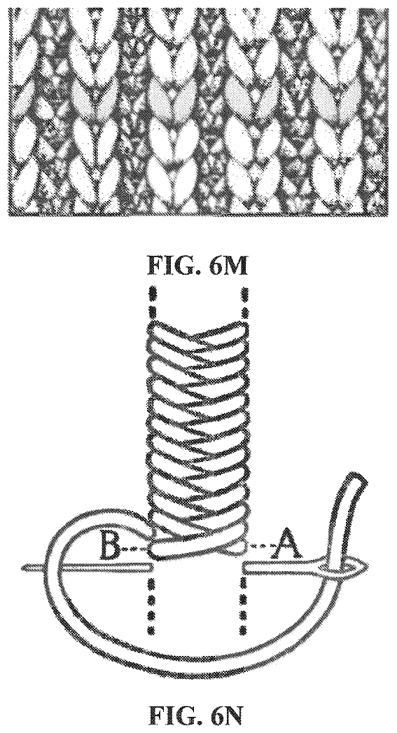

The fabric of the liner can be constructed in a number of ways so as to provide the required functionality. The functionality can be altered by using different yarns or deniers, different elastomers, different weights of elastomers, and different stitches, among other ways, to provide the right longitudinal and circumferential elongation along the length of the liner. Typical yarn types include, but are not limited to, polyester, nylon, acrylic, cellulosic, aramid, natural fibers, and metal wires. Typical elastomers include Lycra/polyurethane, natural rubber, nitrile, and silicone. Typical stitch types for reduced elongation, as shown in FIGS. 6A-6N, include weft (FIG. 6A), warp (FIG. 6B), stockinette (FIGS. 6C and 6D), garter (FIG. 6E), seam (FIG. 6F), fagoting (FIG. 6G), tricot (FIGS. 6H and 6I), elongated (FIG. 6J), plated (FIG. 6K), slip (FIG. 6L), dip (FIG. 6M), and basket (FIG. 6N) and are used solely in the distal region 50 of the liner 100 so as to differentiate the elongation found in the proximal region 60.

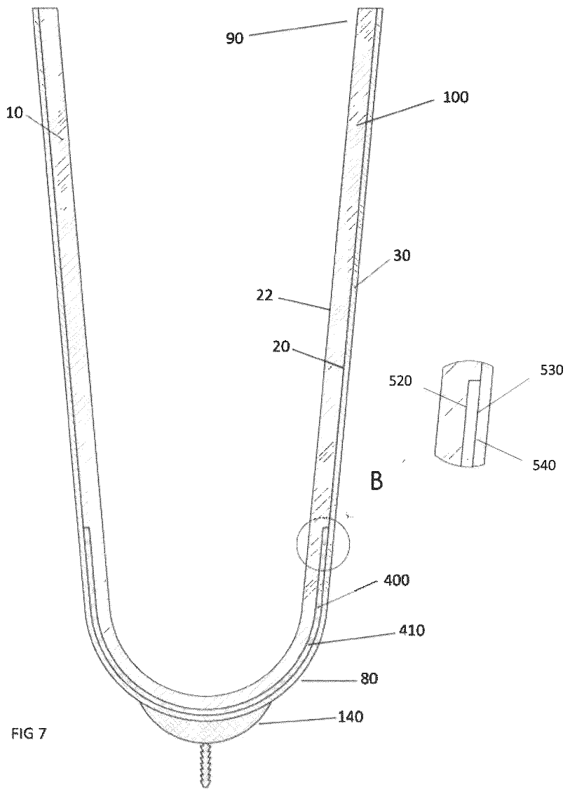

The present invention further incorporates a low extensibility material 400 for use with prosthetic or orthotic devices using the knit design described above. As shown in FIG. 7, this low extensibility material 400 can be used with a prosthetic liner 100 having a distal attachment plate 140 at a distal end 80. The low extensibility material 400 for use with a prosthetic or orthotic assembly preferably comprises a strip of material 410 preferably made of non-elastic materials like carbon fiber, thermoplastics, and polyester on an interior face 520 and a urethane backing 530 on an exterior face 540. Alternatively, the interior face 520 can be made of strips of silk, fiberglass cloth, and other non-elastic materials. The prosthetic liner 100 comprises an elastomer layer 10 having a limb face 22 and a fabric face 20 wherein the fabric face 20 of the elastomer layer 10 is adhered to the external fabric 30 of the prosthetic liner 100. The elastomer layer 10 may be silicone, a styrene-based elastomer such as SEEPS, or any other thermoplastic elastomer now known or to be discovered in the art. In relation to the prosthetic liner 100, the low extensibility material 400 can be adhered to the fabric 30 either internally or externally. If internal placement is desired, the low extensibility material 400 is placed between the elastomer layer 10 and the fabric 30 in the desired region and adhered only to the fabric 30 as shown in the exploded view Part B, forming a composite. Preferably, the low extensibility material 400 is placed at the distal end 80 of the prosthetic liner 100 and extends towards the proximal end 90 of the prosthetic liner 100. The low extensibility material 400 preferably extends upward towards the proximal end 90 of the prosthetic liner 100, stopping at least an inch from the proximal end 90, when placed at the distal end 80. The low extensibility material 400 preferably extends from the distal end 80 about 4-12 inches, preferably 5-7 inches, and is 8-24 inches in total length, preferably 10-14 inches, and 0.5-4 inches in width in order to control distal distraction Similarly, the width of the low extensibility material 400 is between 0.5-4 inches such that it can be placed in a variety of locations and positions. For reference, a prosthetic liner 100 is generally about 18 inches in length prior to any modification made by a prosthetist or patient.

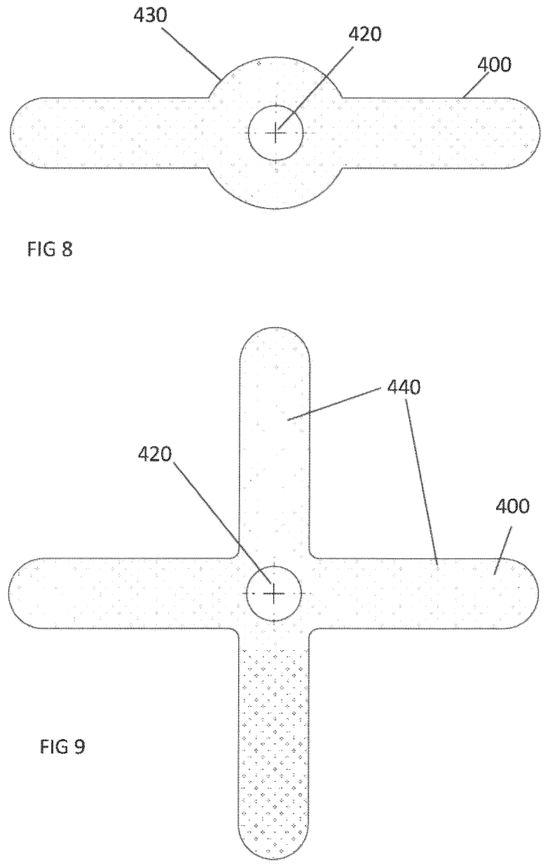

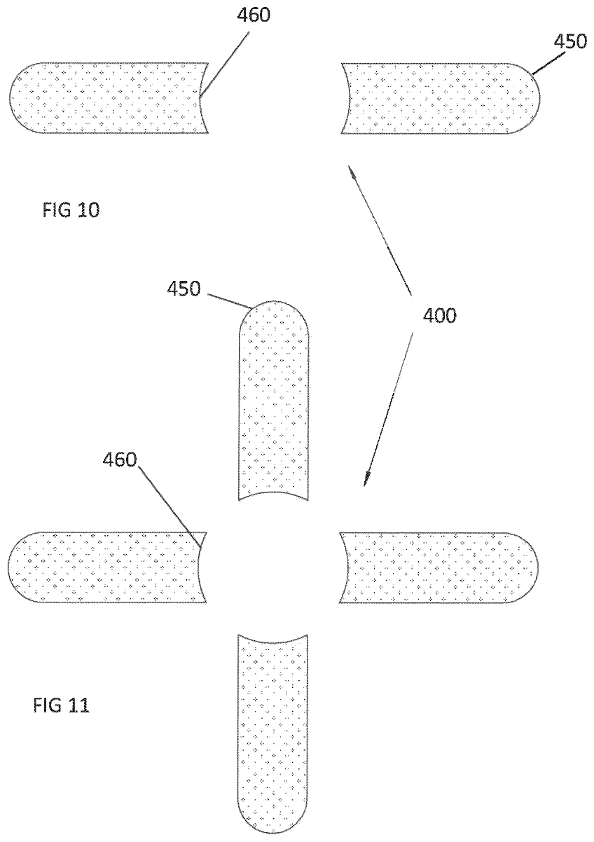

FIGS. 8-13 show the low extensibility material 400 is a variety of configurations. FIG. 8 shows the low extensibility material 400 as a strip of material having a central aperture 420 with a bulged mid-region 430. The locking mechanism 130, as shown in FIG. 7, can be fed through the central aperture 420 if the low extensibility material 400 is adhered externally to the fabric 30. As shown in FIG. 9, the low extensibility material 400 can also be configured to have multiple arms 440 extending radially outward from the central aperture 420. As shown in FIGS. 10 and 11, the central aperture 420 is optional. The low extensibility material 400 can be shaped such that a distal strip end 450 is convex while a proximal strip end 460 is concave. This allows for the low extensibility material 400 to be placed around the distal attachment plate 140 if desired or around the user's joint without impinging on joint movement.

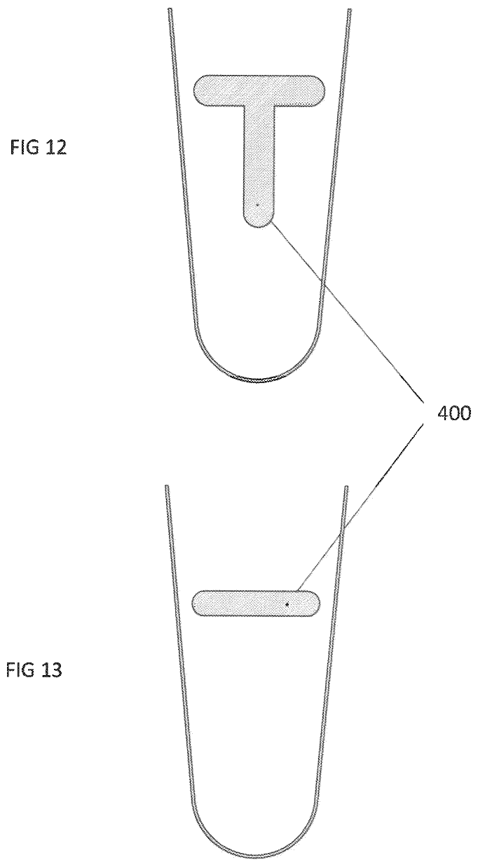

As seen in FIGS. 12 and 13, the low extensibility material 400 can be placed in a variety of configurations according to the needs of the user. The T-shape and single strip configuration shown are not the only configurations that would be helpful to a user and any configuration determined by a medical professional to be medically helpful can be used.

Use of low extensibility material 400 as described herein has been tested and conclusively shows that use of the low extensibility material 400 limits the longitudinal stretch of the liner 100 while maintaining transversal stretch. In the first table, transversal stretch was tested:

| | Reinforced | | | | | Not Reinforced | | | |
|---|---|---|---|---|---|---|---|---|---|
| | before | after | change | % | | before | after | change | % |
| 1 | 13 | 21 | 8 | 61.53846 | 1 | 13 | 20.375 | 7.375 | 56.73077 |
| 2 | 13 | 22 | 9 | 69.23077 | 2 | 13 | 21.5 | 8.5 | 65.38462 |
| 3 | 13 | 20.125 | 7.125 | 54.80769 | 3 | 13 | 22 | 9 | 69.23077 |

| | | | |
|---|---|---|---|
| Average | 61.85897 | Average | 63.78205 |

As seen in the table above, “before” refers to the liner in a static state, meaning no weight was applied, and “after” refers to the liner at failure (i.e. until the strip tore). The number measured is circumference in inches. As can be seen from the test data, the transversal stretch was essentially the same whether the liner was reinforced with the low extensibility material 400 or not.

A similar test was performed testing the longitudinal stretch:

| | Reinforced | | | | | Not Reinforced | | | |
|---|---|---|---|---|---|---|---|---|---|
| | before | after | change | % | | before | after | change | % |
| 1 | 4.25 | 4.5 | 0.25 | 5.882353 | 1 | 4 | 4.75 | 0.75 | 18.75 |
| 2 | 4.25 | 4.5 | 0.25 | 5.882353 | 2 | 4.25 | 4.75 | 0.5 | 11.76471 |
| 3 | 4.5 | 4.75 | 0.25 | 5.555556 | 3 | 4.25 | 4.625 | 0.375 | 8.823529 |

| | | | |
|---|---|---|---|
| Average | 5.77342 | Average | 13.11275 |

As can be seen from this test, the longitudinal stretch was reduced by more than half when the low extensibility material 400 was used.

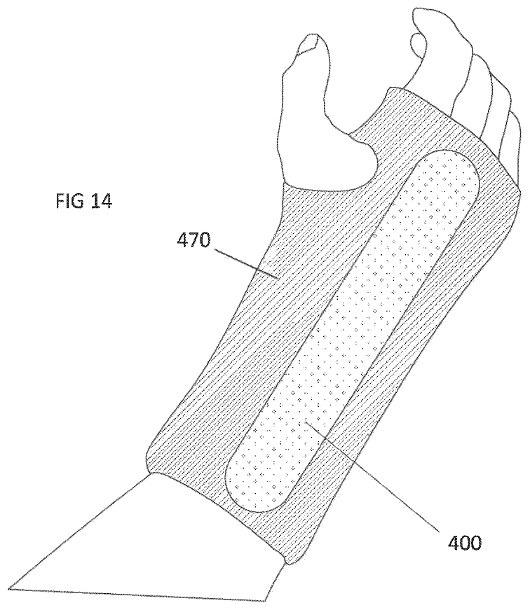

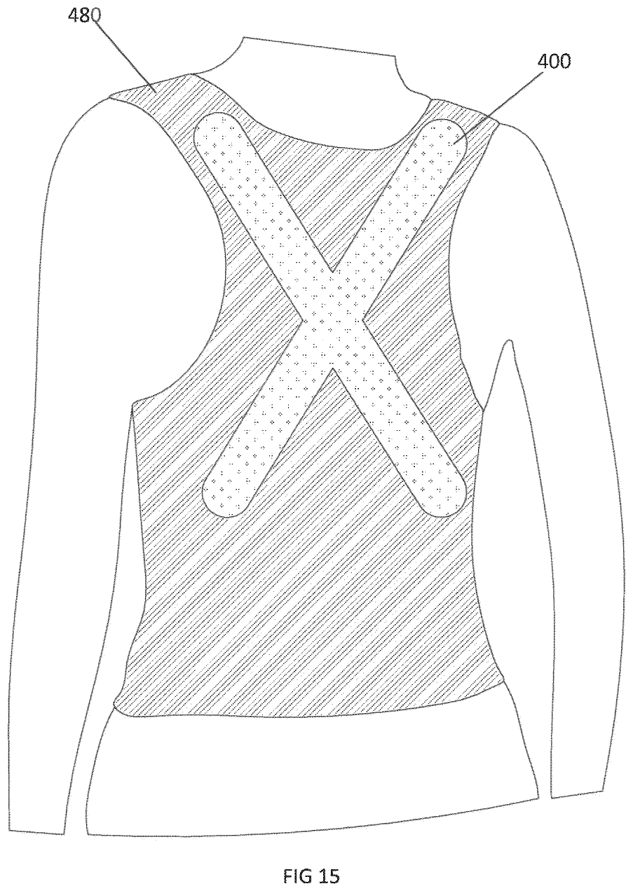

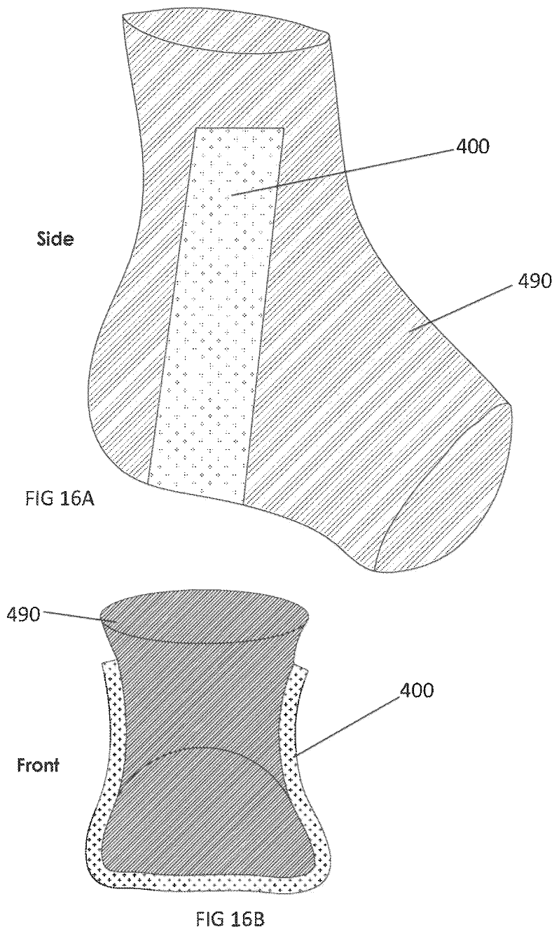

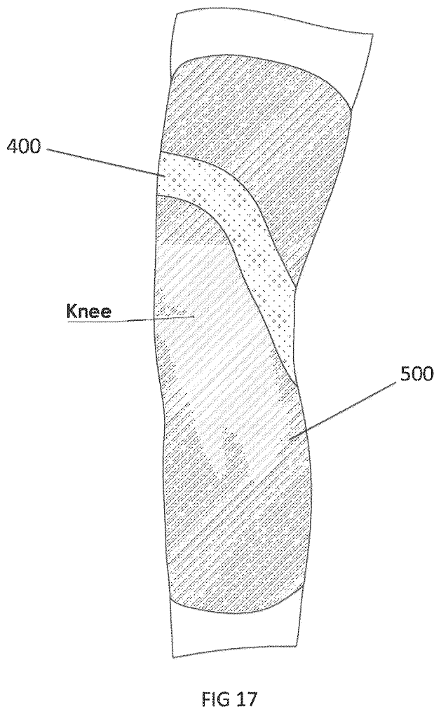

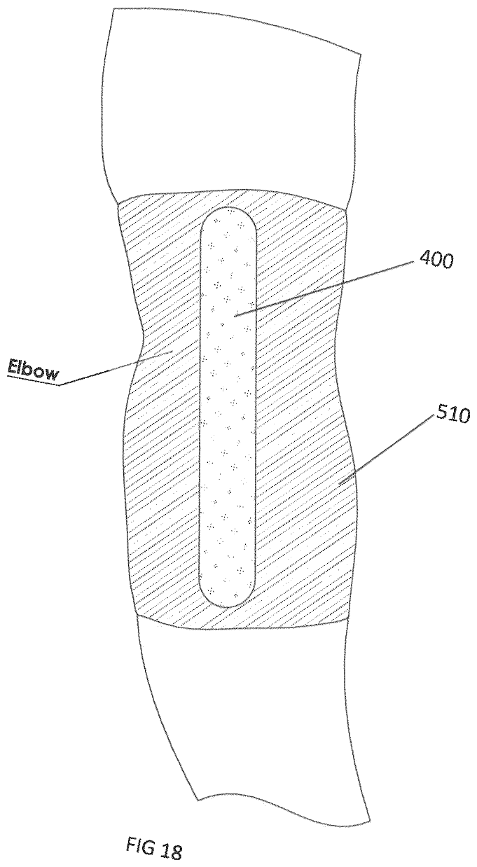

The low extensibility material 400 can also be used with orthotics which is useful to limit joint flexion both as a therapeutic aid in the case of injuries or as a means of injury prevention. As seen in FIG. 14, the low extensibility material 400 works with a wrist brace 470 and can be adhered externally or internally as described above. Similarly, as shown in FIG. 15, the low extensibility material 400 can be integrated with a back brace orthotic 480. Likewise, the low extensibility material 400 can be used with an ankle orthotic 490, as shown in FIGS. 16A and 16B, where the low extensibility material 400 preferably has a thickness to prevent ankle movement. FIG. 17 depicts the use of the low extensibility material 400 with a knee orthotic 500 and FIG. 18 depicts the use of the low extensibility material 400 on an elbow orthotic 510. In all configurations when in use with an orthotic, the low extensibility material 400 can be in a strip or in a specific shape, as determined by an orthotic physician, and be adhered to the fabric 30 either internally or externally.

When the low extensibility material 400 is desired to be used internally, the low extensibility material 400 is first adhered to the fabric 30 on the fabric face 20 using conventional means and then the combination is placed in a mold wherein the gel, silicon, or urethane is molded over, creating a composite. Alternatively, exterior use is performed by adhering the low extensibility material 400 onto the outside of the fabric 30 in the desired location.

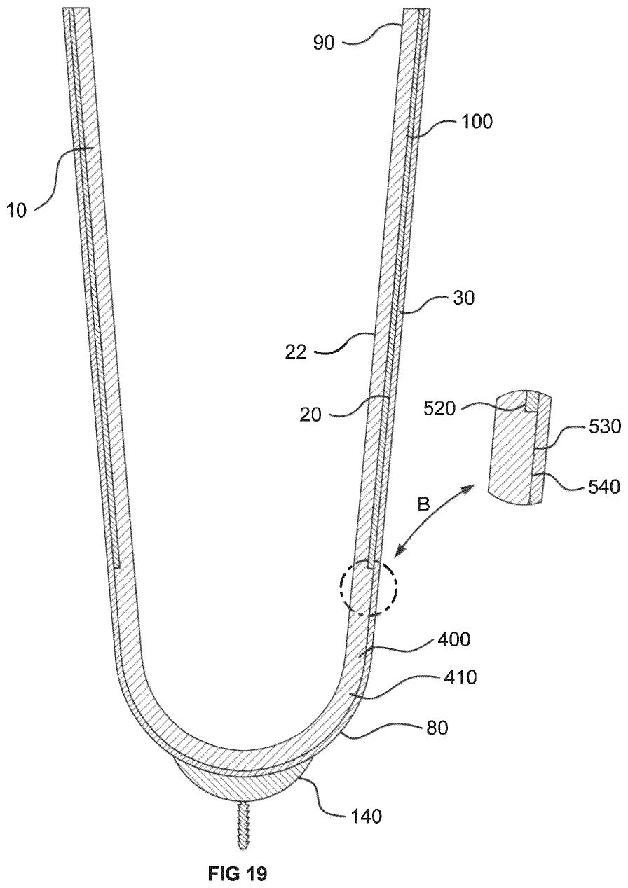

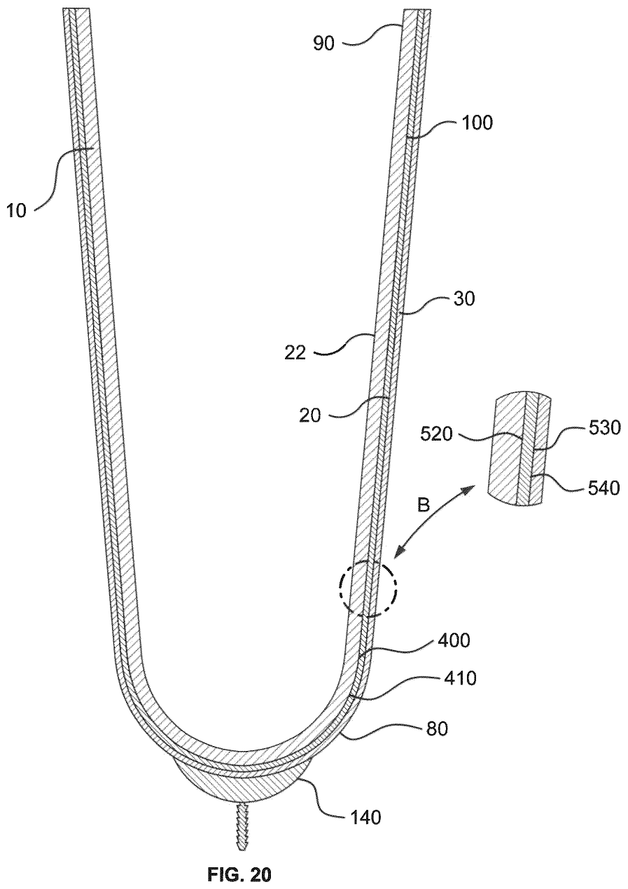

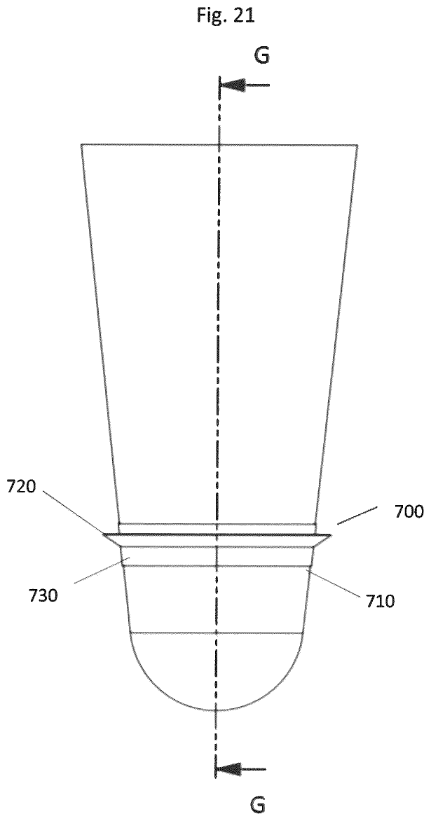

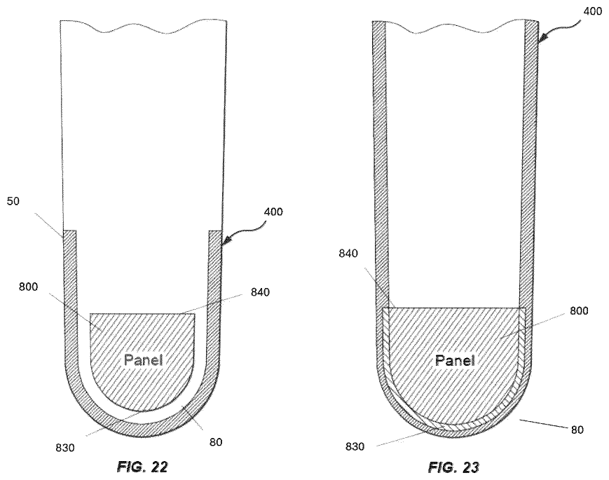

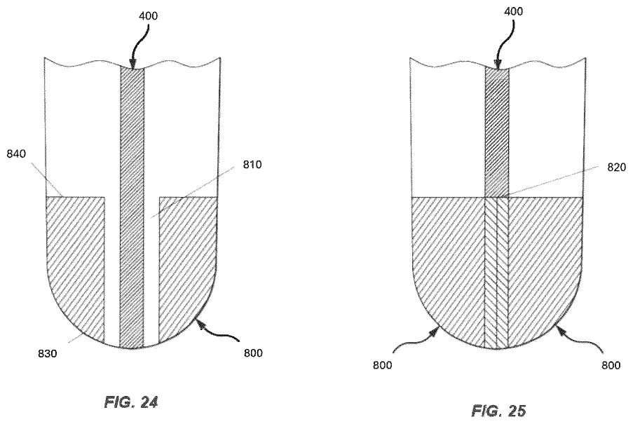

Alternatively, as shown in FIG. 19, the low extensibility material 400 is adhered internally from the distal end 80 up to the upper edge 520 of the proximal end 90 meaning there is no portion of the liner 100 that does not have limited longitudinal stretch due to reinforcement. In another alternative embodiment, as shown in FIG. 20, the low extensibility material 400 is adhered from the upper edge 520 distally to the top of the distal region 50. In addition, a sealing element 700 may be affixed to the exterior fabric layer 30, as described in U.S. patent application Ser. No. 18/221,522, the contents of which are incorporated by reference. The sealing element 700 can be made of either silicone or an elastomeric compound such as urethane, which is non-unitary with either the exterior fabric layer 30, through use of a bond layer 710. Preferably the bond of the bond layer 710 is a type of glue which creates a seal thereby forming a vacuum. The sealing element 700 further comprises an engaging member 720 which extends radially outwardly from the sealing element base 730.

As shown in FIGS. 22-25, an alternative embodiment is shown incorporating at least one distal panel 800. The distal panel 800 is preferably made of the same material as the low extensibility material 400 but may differ. The distal panel 800 can be made of carbon fiber, a thermoplastic, strips of silk, polyester, or a similar inelastic material. The panel 800 is located at the distal end 80 and extends up to the top of the distal region 50. The distal panel 800 is preferably hemispherical in that the distal panel 800 does not wrap around the entirety of the distal end 80 but can leave a gap 810, as seen in FIG. 24, or can meet at a midpoint 820 where the low extensibility material 400 is located, as seen in FIG. 25. Likewise, as seen in FIG. 23, the distal panel 800 may cover only a portion of the distal end 80. The distal panel 800 has a rounded distal panel end 830 and a flat proximal panel end 840 to conform to the shape of the distal end 80.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A prosthetic liner comprising:
a fabric covering having an open proximal end and a closed distal end knit into a tubular shape, the fabric covering further comprising a proximal region and a distal region wherein a distal region having a terminal edge is stitched such that it has a distal longitudinal elongation of 0 to 30% from a resting position and wherein a proximal region has a proximal longitudinal elongation of 55-125% from a resting position;
wherein the distal region is stitched using a distal stitching selected from the group consisting of weft, warp, stockinette, garter, seam, fagoting, tricot, elongated, plated, slip, dip, or basket stitches;
an styrene-based elastomeric gel layer residing on an interior surface of the fabric covering;
at least one hemispherical distal panel located at the closed distal end; and,
a single-piece low-extensibility strip comprising polyester forming a composite with the styrene-based elastomeric gel layer and the fabric covering extending up from the closed distal end to an edge of the open proximal end and having a width between 0.5 and 4 inches.

2. The prosthetic liner of claim 1 wherein the single-piece low extensibility strip further comprises urethane facing the fabric covering.

3. The prosthetic liner of claim 1 wherein the distal region has a distal horizontal stretch of 10-200% from a resting position.

4. The prosthetic liner of claim 1 wherein the proximal region has a proximal horizontal stretch of 100-175% from a resting position.

5. The prosthetic liner of claim 1 further comprising an intermediate region having an intermediate longitudinal elongation of 15-40% and a horizontal elongation of 80-120%.

6. The prosthetic liner of claim 1 wherein the terminal edge of the distal region is at least four inches from the closed distal end.

7. The prosthetic liner of claim 1 further comprising two hemispherical distal panels meeting at a midpoint.

8. The prosthetic liner of claim 1 further comprising two hemispherical distal panels that do not meet and leave a gap where the single-piece low-extensibility strip is located.

9. A prosthetic liner comprising:
a fabric covering having an open proximal end and a closed distal end knit into a tubular shape, the fabric covering further comprising a proximal region and a distal region wherein a distal region having a terminal edge is stitched such that it has a distal longitudinal elongation of 0 to 30% from a resting position and wherein a proximal region has a proximal longitudinal elongation of 55-125% from a resting position;
wherein the distal region is stitched using a distal stitching selected from the group consisting of weft, warp, stockinette, garter, seam, fagoting, tricot, elongated, plated, slip, dip, or basket stitches;
an styrene-based elastomeric gel layer residing on an interior surface of the fabric covering;
at least one hemispherical distal panel located at the closed distal end; and,
a single-piece low-extensibility strip comprising polyester forming a composite with the styrene-based elastomeric gel layer and the fabric covering extending up from the terminal edge to an edge of the open proximal end and having a width between 0.5 and 4 inches.

10. The prosthetic liner of claim 9 wherein the single-piece low extensibility strip further comprises urethane facing the fabric covering.

11. The prosthetic liner of claim 9 wherein the distal region has a distal horizontal stretch of 10-200% from a resting position.

12. The prosthetic liner of claim 9 wherein the proximal region has a proximal horizontal stretch of 100-175% from a resting position.

13. The prosthetic liner of claim 9 further comprising an intermediate region having an intermediate longitudinal elongation of 15-40% and a horizontal elongation of 80-120%.

14. The prosthetic liner of claim 9 wherein the terminal edge of the distal region is at least four inches from the closed distal end.

15. The prosthetic liner of claim 9 further comprising two hemispherical distal panels meeting at a midpoint.

16. The prosthetic liner of claim 9 further comprising two hemispherical distal panels that do not meet and leave a gap where the single-piece low-extensibility strip is located.
